(19) United States
(12) Patent Application Publication
(10) Pub. No.: US 2004/0015130 A1
(43) Pub. Date: Jan. 22, 2004

(54) Vein retaining device

(76) Inventors: Calvin Lee Neumann, Key West, FL (US); Leslie Louise Neumann, Winston-Salem, NC (US)

Correspondence Address: Calvin L. Neumann, 1224 Huntingdon Rd, Winston. Salem, NC, 27104, US

(21) Appl. No.: 10/196,630

(22) Filed: Jul. 16, 2002

(51) Int. Cl. A61M 5/00

(52) U.S. Cl. 604/116

(57) ABSTRACT

A medical vein retaining device with two or more edges which assists in the initiation of an intravenous infusion. The device is placed on the skin with the edges on each side of the vein to be infused, which prevents the vein from sliding or rolling away from the hypodermic needle when initiating an intravenous infusion.

### CROSS REFERENCE TO RELATED APPLICATIONS

Not applicable

### FEDERALLY SPONSORED RESEARCH

Not applicable

### SEQUENCE LISTING OR PROGRAM

Not applicable

### BACKGROUND OF THE INVENTION—FIELD OF INVENTION

This invention relates to medical devices, specifically to intravenous infusion aids and supports.

### BACKGROUND OF THE INVENTION

Intravenous infusions or connections are a standard procedure for the administration of drugs and the giving of blood and blood products to subjects by health practitioners. Such intravenous connections are also used when blood is withdrawn from subjects. Intravenous infusions almost always require a skilled health professional to initiate the intravenous connection. When an intravenous infusion is initiated on a subject, the procedure is usually as follows: first, a tourniquet is applied between the intravenous site selected and the heart. Next, the site is cleansed and the vein is allowed to reach full turgor. Then, the hypodermic needle, connected by tubing to a syringe with sterile saline solution, is inserted through the skin, through the surface of the vein and into the vein. Finally, the hypodermic needle is fixed in place with tape, the tourniquet is removed, saline solution is injected into the vein to check the success of the initiation, and then the needle and tubing is connected to the supply of drugs or whatever is to be administered to the subject.

In this procedure, many subjects will have veins lying close to the surface of the skin, and while these veins are easy to see, they often slide or roll under the skin away from the point of the hypodermic needle, thus making the initiation of the intravenous infusion difficult. This can result in a very difficult or even unsuccessful intravenous infusion initiation, subcutaneous hemorrhaging and considerable trauma to the subject.

Current medical practice is to use an intravenous infusion site on a subject for three days, then to initiate another intravenous infusion at another site for another three days. New infusion sites must take into account local trauma such as bruising or subcutaneous hemorrhaging. Thus the successful initiation of an intravenous infusion requires skill and practice, and veins that slide or roll cause difficulty in this procedure and possible trauma to the subject.

While many inventions address the needs of intravenous infusions, with devices to hold hypodermic needles in place after insertion, improved hypodermic needles, and devices to hold arms and hands rigid, none of these devices address the need to assist in the initiation of an intravenous infusion by preventing the sliding or rolling of veins away from the hypodermic needle. For instance, U.S. Pat. No. 6,042,568 to Gomez discloses a patient mounted I/V protector used to prevent the I/V needle from being interfered with after the I/V connection is made. Other devices also support or protect intravenous (I/V) connections after they have been initiated, such as U.S. Pat. No. 5,076,298 to Darling and U.S. Pat. No. 6,015,119 to Starchevich, but again, do not deal with the problems and difficulties of initiating an intravenous infusion. U.S. Pat. No. 5,509,902 to Raulerson discloses a catheter stabilizing device, but again, this device is not for use when initiating an intravenous infusion, but for subcutaneously stabilizing and securing an indwelling catheter.

Improved, needles are shown, for example, in the Introcan®—W Safety™ I/V Catheter made by B. Braun Medical Inc., Bethlehem, Pa. which is designed to minimize inadvertent needle sticks. Here a flexible plastic catheter is fitted over the hypodermic needle. The needle is inserted into the vein and then the flexible catheter is threaded into the vein and the needle is removed. This minimizes punctures through the bottom of the vein, but does not assist in the initiation of the intravenous infusion with respect to veins that slide or roll away from the hypodermic needle.

Other devices, such as disclosed in U.S. Pat. No. 4,982,744 to Stanec, address ways to immobilize the subjects forearm or hand in administering an intravenous infusion, but do not address ways to prevent veins from sliding or rolling away from the hypodermic needle when the intravenous infusion is initiated.

Thus, it can be seen that even though rolling or sliding veins present a problem when starting an intravenous infusion, there are no devices or methods which help this problem.

### SUMMARY

It has been discovered that a small device may be used to prevent the sliding or rolling of veins away from the hypodermic needle, and thus render the initiation of an intravenous infusion a much simpler, less traumatic, safer and easier process.

### DRAWINGS—FIGURES

FIG. 1 shows a perspective view of a vein retaining device with two edges, one connecting, and one hook.

FIG. 2 shows a perspective view of a vein retaining device made of wire with four edges and two connecting members.

FIG. 3 shows a perspective view of a vein retaining device with two edges, one wide connecting member, and a notch in the connecting member.

FIG. 4 shows a perspective view of a vein retaining device with two edges and one connecting member.

### DRAWINGS—REFERENCE NUMERALS

| | | |
|---|---|---|
| 10 edges, | 12 hook | 14 connecting member |
| 20 edges | 22 low portion | 24 connecting member |
| 30 edges | 32 notch | 34 connecting member |
| 40 edges | | 44 connecting member and saddle |

### DETAILED DESCRIPTION

This discovery is a device having two or more edges in such juxtaposition that the device may be placed over the skin and turgid vein with at least one edge on each side of the vein, and a space between the edges large enough to allow the vein to remain turgid and ready for an intravenous infusion initiation, but with the edges close enough to prevent the vein from sliding or rolling away from the hypodermic needle. Because the vein can no longer slide or roll away from the hypodermic needle, intravenous infusion initiation becomes a much simpler and easier process. Further, the chances of trauma and an unsuccessful initiation are greatly reduced.

FIG. 1 shows such a vein retaining device with two essentially parallel edges and a connecting member that is a bridge. The device is made of wire or other similar malleable material. In the practice of this invention, using the specific embodiment shown in FIG. 1 as an example but not as a limitation, the edges 10, are placed on the skin either side of the vein to be infused, with the connecting wire bridge 14, above the vein. The vein retaining device may be conveniently held in place with finger pressure on the connecting bridge 14, or with a rubber band, tape or other ligature on the hook 12. The downward pressure on the connecting bridge or on the hook holds the tines or edges in place on the skin over the vein and the hypodermic needle is easily inserted in the space between the tines.

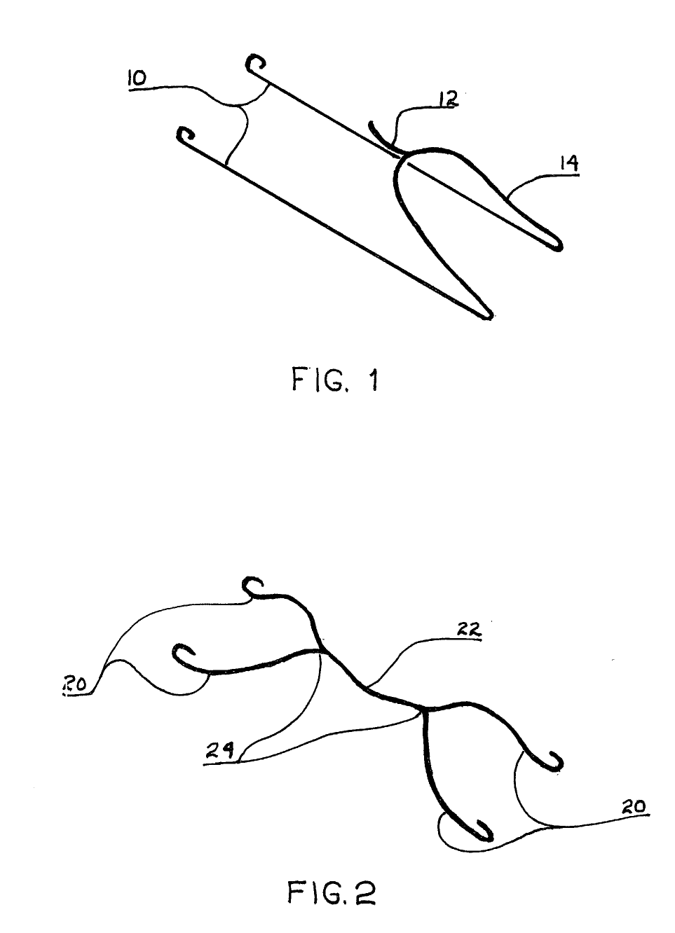

Another useful embodiment of this invention is shown in FIG. 2, where the device is made of a malleable material such as wire, especially stainless steel wire. The embodiment of the vein retaining device shown in FIG. 2 has four edges 20 and is utilized by placing two edges on each side of the vein. The connecting members 22 in the center include a low portion 24 so the device may be easily held in place with finger pressure, a rubber band, string, or other ligature. Moreover, because the vein retaining device is made of a malleable material, it may easily be adjusted in width for use on veins of different sizes.

Other embodiments of this invention may be manufactured such that the connection is able to articulate in order to accommodate veins of different diameters. As an example, but not as a limitation, when the device shown in FIG. 2 is made from two pieces of wire which are twisted together in the center low portion 24 then the device may articulate at this twisted connection and thus be adjusted in width at that articulation for use on veins of different diameters.

FIG. 3 shows another specific embodiment of this invention. Here the vein retaining device is made in a cylindrical fashion with the bottom edge of the cylinder removed to make two essentially parallel edges 30 The edges 30 are placed on the skin either side of the vein prior to the initiation of the intravenous infusion. A notch 32 is in the upper center portion of the device to conveniently receive a rubber band or other ligature, and the end of the device is made with its leading edges extended so that the hypodermic needle may be easily inserted into the vein.

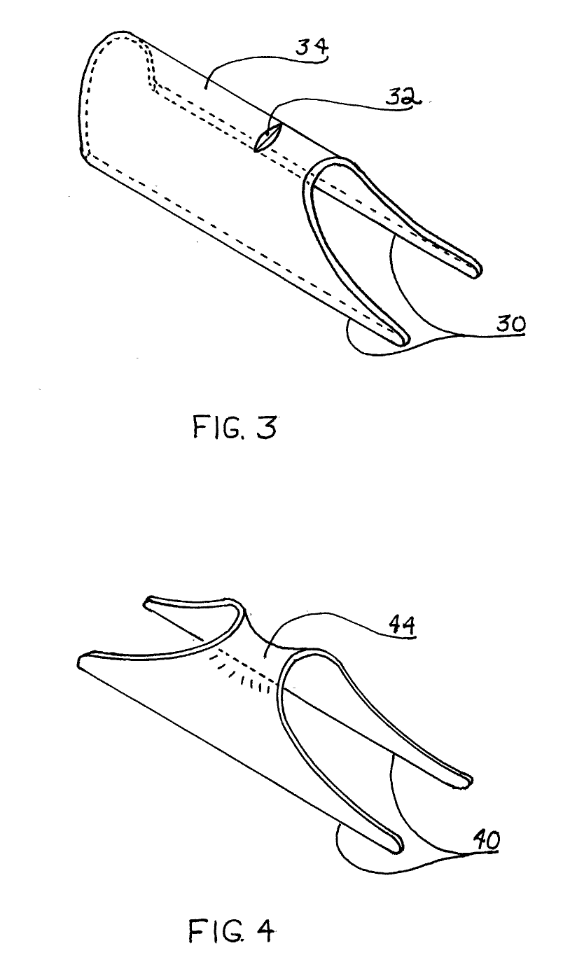

FIG. 4 shows a preferred embodiment of this invention. Here the vein retaining device is made in a cylindrical fashion with the bottom edge of the cylinder removed and the leading and trailing edges extended, resulting in a device with two edges 40 and a connecting member 44 in the middle. The connecting member 44 is constructed with a low area or saddle so that it may be held in place with finger pressure, a rubber band, or other ligature. This vein retaining device is manufactured from malleable sheet metal or flexible plastic, and therefore easily adjusted in width.

Still other embodiments of this invention may be manufactured without a connecting member by simply connecting the edges together at their ends. A vein retaining device made in this manner from four wire edges joined in the center would have an “X” shape with the center of the “X” raised enough to clear the vein.

Still other embodiments of this invention may be manufactured with attaching means such hooks or loops to hold the vein retaining device in place on the skin over the vein with a rubber band or ligature. The attaching means may be on the connecting member, on the edges, or both.

Still other embodiments may be manufactured from flexible plastic materials such as nylon, polyethylene, polystyrene, polycarbonate or the like.

Still other embodiments of this invention may be manufactured from combinations of material such as plastic coated wire, plastic tipped wire, wire reinforced plastic, composites such as fiberglass reinforced plastic, and other materials too numerous to detail.

The vein retaining device described herein is of a size such that it may be placed over the skin with the edges either side of the vein, but will not compress the vein unduly, but not so large as to allow the vein to move away from the hypodermic needle. This results in a device of approximate dimensions of 2.0-2.5 cm long, 0.5-1.2 cm wide and 1.0-1.3 cm high. It is obvious that larger or smaller devices may be manufactured for use on larger or smaller veins and still lie within the spirit and scope of this invention.

Further, it is obvious that this device is useful for intravenous infusions on animal as well as human subjects, and may vary greatly in size for use on large or small animals, still being within the scope and spirit of this invention.

An unexpected and useful result of this invention is that it is particularly advantageous when an intravenous self-infusion is given. When a subject gives himself or herself an intravenous infusion, it not only requires skill and training, but also requires normally that a medical device called a Port-a-cath be surgically implanted. The Port-a-cath consists of a rubber diaphragm connected to a catheter. The Port-a-cath is surgically implanted with the diaphragm just under the skin and the catheter end of the device connected to a major vein. While this device allows the self-infusion to be performed by needle insertion through the skin into the diaphragm using two hands, it results in a number of problems. It must be flushed regularly, and there is a high incidence of infection associated with it's use. Additionally, the trauma and risk of a surgical implant is not trivial. With the vein retaining device disclosed in this patent, only one hand is required for initiation. The vein retaining device described herein may be placed over the vein at the infusion site and held in place with a rubber band or other ligature. The intravenous connection is then easily initiated. The vein retaining device may then be simply removed by cutting the rubber band or other ligature, and thus only one hand is required for the entire procedure. The vein retaining devices shown in FIGS. 1, 2, 3 and 4 are particularly useful in this respect, as the hook in the center or the device shown in FIG. 1, the low area in the center of the device shown in FIG. 2, the notch in the center of the device shown in FIG. 3, and the saddle in the center of the device shown in FIG. 4 allow the ligature to remain in place without finger pressure, thus freeing the hand for the task of hypodermic needle insertion.

The term articulated in the description of this invention means moveable by mean of joints, hinges or the like.

The term vein in the description of this invention is meant to include all blood vessels, including veins, venules, arteries, arterioles and capillaries. While it is understood that the vast majority of infusions or connections to the blood supply of a subject occur at veins, it is obvious that under some circumstances, venules, arteries or arterioles may also be used.

The term intravenous infusion, in the description and within the spirit and scope of this invention, is meant to include all forms of hypodermic blood vessel connections, for whatever purpose, so long as a hypodermic needle is used to initiate the connection, whether for the administration of drugs, fluids, medicines, placebos, blood withdrawal, and blood or blood product transfusions and infusions.

In conclusion, the vein retaining device described herein provides a means and method to simplify the intravenous infusion process, particularly when subjects self-infuse. This results in increased safety, and minimizes trauma to the subject by preventing the vein from sliding or rolling away from the hypodermic needle during the initiation of an intravenous infusion.

We claim:

1. A device to aid in the initiation of an intravenous infusion with at least two edges in such juxtaposition that said device may be placed with at least one of said edges on each side of a vein and thus prevent the vein from sliding or rolling away from the hypodermic needle.

2. A device which prevents the rolling or slipping of a vein away from the hypodermic needle during the initiation of an intravenous infusion comprising:
(a) at least two edges,
(b) at least one connecting member between said edges.

3. A device as claimed in claim 2 where said edges are essentially parallel.

4. A device as claimed in claim 2 with said connecting members placed so that pressure on said connecting members hold the edges in place on the skin over the vein.

5. A device as claimed in claim 2 able to be held in place on the skin over the vein with finger pressure, a rubber band or other ligature.

6. A device as claimed in claim 2 further including a means to attach a rubber band or other ligature to hold said device in place on the skin over the vein.

7. A device as claimed in claim 2 where the size of said device is such that it may be placed over the skin with said edges either side of the vein, but will not compress the vein unduly, and is not so wide as to allow the vein to move away from the hypodermic needle.

8. A device as claimed in claim 2, which may be adjusted to the width of the vein.

9. A device as claimed in claim 2 made of a malleable or flexible material.

10. A device as claimed in claim 2 further including an articulated connection whereby the distance between said edges is able to be adjusted in width.

11. A device as claimed in claim 2 approximately 2.0-2.5 cm long, 0.5-1.2 cm wide, and 1.0-1.3 cm high.

12. A device as claimed in claim 2 where said device is made of wire.

13. Devices as claimed in claim 2 as shown in FIGS. 1, 2,3 and 4.
